(19) United States
(12) Patent Application Publication
(10) Pub. No.: US 2026/0297484 A1
(43) Pub. Date: Oct. 1, 2026

(54) PNEUMATIC MICROFLUIDIC PERFUSER FOR CELL CULTURE

(71) Applicant: National Research Council of Canada, Ottawa (CA)

(72) Inventors: Pun Pang SHIU, Ottawa (CA); Byeong-Ui MOON, Brossard (CA); Keith MORTON, St-Bruno-de-Montarville (CA); Teodor VERES, Montreal (CA)

(73) Assignee: National Research Council of Canada, Ottawa (CA)

(21) Appl. No.: 19/633,596

(22) Filed: Mar. 30, 2026

(30) Foreign Application Priority Data
Mar. 31, 2025 (GB) 2504831.5

(51) Int. Cl. C12M 3/06; C12M 1/32; C12M 1/42

(52) U.S. Cl. CPC C12M 23/16; C12M 23/12; C12M 35/04

(57) ABSTRACT

A perfuser for microfluidic cell culturing comprises a relief-patterned substrate defining a plurality of perfusion channels between paired medium exchange receptacles, each receptacle having: a floor; a medium outlet at or near the floor; a closed array of least one sidewall separating the floor from a first relief-patterned surface of the substrate; and a gravitationally checked microvalve (GCM) for feeding medium into the receptacle at an opening of the GCM. The perfusion channels each having a hydraulic diameter (d_{HC}) of 0.1-1 mm for transport of the medium to a respective interface of a 3-raceway cell culture layer that is part of, or designed for sealed meeting with, the perfuser. A separation between the opening and the floor is at least 5 mm. Also provided are kits comprising said devices and methods for operating said kits.

### FIELD

The present disclosure relates, in general, to microfluidic biological assays and cultures, and in particular to a patterned microfluidic substrate for perfusing biological culturing of cells and/or tissues, including organ-on-chip (OOC) devices.

### BACKGROUND

Amongst the many biological studies of living matter (typically having cells, and preferably comprising one or more cells of a first type, a scaffold including some extracellular matrix (ECM), and one or more cells of a second type, or organelles, viruses, organoids or other discrete biological structures), are models of blood vessels. Blood vessels in humans and animals form a complex network that is crucial for transporting blood and cells, delivering oxygen, nutrients, and removing cell-produced metabolic waste and carbon dioxide, to support functions of every organ and tissue throughout the body. The inner lining of blood vessels consists of a single layer of endothelial cells, which acts as a barrier between the bloodstream and surrounding tissues. Shear stress is a critical factor in maintaining endothelial function, vascular homeostasis, regulating blood flow, and angiogenesis (Hui Lin Zhou et al, “Role of blood flow in endothelial functionality: a review” (2023), Front. Cell Dev. Biol. 11:1259280). To mimic physiologically relevant conditions in microfluidic organ-on-chip (OOC) and culturing devices that aim to replicate organ-level functions, the ability to precisely control and stimulate fluid flow is of critical importance.

Perfusion flow also plays a critical role in a host of cell culturing devices, where two or more organ cell types (or one organ cell type and one or more other biologically relevant discrete biological structures) are co-cultured or enabled to flourish in controlled proximity to allow dynamic interactions between them, thereby providing conditions more representative of human physiology. By mimicking physiological fluid flow, active control perfusion (perfusion that varies in time, or in response to certain activities) may permit more realistic inter-cell, inter-tissue, or inter-organ communication, offering significant insights into complex biological processes. These enabled interactions allow one to study inter-organ toxicity, gene regulation, and drug-drug interactions of cells and tissues in a controlled microenvironment. Additionally, such systems can simulate the effects of drugs metabolized by cells, tissues, and organs on one another and in the larger biological system, leading to more accurate predictions of systemic effects into the other organs. (Paul M. van Midwoud et al, Lab Chip, 2010, 10, 2778-2786. DOI: https://doi.org/10.1039/C0LC00043D; Leung et al, 2022, “A guide to the organ-on-a-chip.” Nature Reviews Methods Primers 2, 33)

Various approaches have been demonstrated to mechanically simulate blood flow, in OOC platforms (Joshua Lim et al., ACS Biomater. Sci. Eng. 2024, 10, 6, 3548-3567, DOI:10.1021/acsbiomaterials.3c01978). These include microfluidic devices in which fluid flow is driven by external forces generated by pumps or gravity, which are not entirely successful in providing physiologically relevant fluid flow to cell cultures.

Syringe pump-based flow systems utilize an actuating mechanical system to push or pull liquids into OOC devices via tubing, offering a simple and efficient experimental setup with set-point controlled flow rates. Small artery segments are incorporated into the OOC platform to assess the structure and function of resistance arteries. Syringe pumps facilitate media delivery and enable drug testing in these setups. (Gunther et al, Lab on a chip, 2010, DOI: 10.1039/C004675B)

Pneumatically-controlled flow systems offer stable and responsive flows, as pressure controllers are able to establish non-pulsatile flows. Supported by a manifold holder, the pressure-controlled system can deliver multiple liquids into the OOC devices, allowing for media transfer to the other side of the microfluidic reservoir (Yasotharan et al., Lab Chip, 2015, 2660-2669. DOI: 10.1039/c5lc00021a).

Gravity-driven hydrostatic flow is also frequently used in OOC devices, controlled by altering the height of the media reservoir, the dimensions of the microfluidic channels, or by manipulating the incline of rocking systems, for example, OrganoFlow® by MIMETAS. (Moon et al., Lab on a chip, 2024, DOI: 10.1039/d4lc00530a).

Traditional 24, 48, 96 and 384-wellplate cell cultures provide reasonable throughput in terms of drug discovery but are severely limited due to non-physiologically relevant flat cell culture and non-physiological cell-cell communications. For example, due to hypoxia (low levels of oxygen in cells), growth of the lower layer of cells that are not in direct contact with cell culture media is limited. Various microfluidic structures for holding, and interacting with (including alimenting) cellular matter for growth can offer new insight.

For physiologically relevant cell culture systems, syringe pumps provide continuous flow with precise and reliable control over fluid delivery, making them essential for ensuring stable and consistent conditions in microfluidic OOC systems. However, a major drawback of syringe pump systems is their limited capacity for multiplexed applications. Their operation typically involves challenges with tubing interfaces (or more generally chip-to-world issues), and limited volume capacities, which in turn requires frequent manual intervention for reloading or managing multiple parallel channels. As a result, scaling up for multiplexed experimentation becomes cumbersome, inefficient, time-consuming, and labour-intensive.

Hydrostatic flow-based OOC devices with their small footprint provide a compact solution for fluid delivery. However, they often face reliability issues due to changes in flow rate over time, as the liquid level decreases, leading to inconsistent flow. Although rocker systems can allow quasi-continuous flow by gently tilting the device to maintain fluid movement, they still lack the precision needed for certain applications. In addition, the rocker takes up significant volumetric space within a cell culture incubator. While advancements have been made to improve throughput, significant challenges remain, particularly when managing a large number of samples or simultaneous experiments, in terms of maintaining consistent flow rates across multiple, different chips in a high-throughput setup difficult. Furthermore such systems have provisioned fluid-dynamic resistances that cannot be varied, and so no change in perfusion rates can be effected in such systems.

Microvalves are an important component of OOCs, which enable delivery of microvolumes of fluids in a sequential and punctuated manner leading to automation of assay protocols. While a few microvalves may resemble microscale versions of macroscale valves, microvalving is generally a distinct field of endeavour, at least in part because surface tension dominates flow behaviour at the microscale, and gravitational force is weak in comparison. Most active microvalves are chip-integrated features in stacked multilayer structures that can be difficult to fabricate and assemble into devices on a large scale, and typically involve competing forces that require more force and energy to deploy and control. This is particularly noticeable in relation to the structures and resiliences required for checking (i.e., backflow prevention). While simpler microvalve structures exist, such as siphon valves, they require machinery to operate, such as centrifuges.

There remains a need for cell culture perfusion devices that are compact and energy efficient, robust, mass-producible, modular and quickly and easily assembled. Furthermore it is desirable to deliver fresh medium, and extract culture media from 3D cell cultures, with minimal interference with perfusion, as well as provide drug dosing. It is also desired to provide unidirectional perfusion where this is physiologically relevant.

This background information is provided for the purpose of making known information believed by the applicant to be of possible relevance to the present disclosure. No admission is necessarily intended, nor should be construed, that any of the preceding information constitutes prior art against the present disclosure.

### SUMMARY

The present disclosure relates to a pneumatic-based cell culture perfuser. The perfuser has a patterned surface providing a plurality of low-pressure seals at two perfusion channels at two opposite ends of each perfusion channel. The perfusion channels flank a central cell culture region along a respective cell culture interface. The perfuser has at least a first and a second three-phase receptacle for each perfusion channel, and a flow path directing cell media from the first receptacle via the interface to the second receptacle through the perfusion channel. The flow path can be half-duplex, to recycle perfusion by directing the media back into the first receptacle via the interface, in some embodiments, and in others, the flow path can bypass the interface in redirecting the fluid into the first receptacle, describing a loop that results in a continuous unidirectional reflow of the same medium. In some embodiments, this flow channel, whether reversing or continuous, has a branch, for either or both of: removing spent medium, injecting fresh medium, sampling and optionally testing medium, or injecting drug, adjuvant, growth moderator, CO2, O2, or other solid, liquid, or gaseous material. The perfuser may be multiplexed. The perfuser, though providing adequate low pressure sealing to allow for liquid to be nearly continuously, or with some frequency, injected and retracted from the perfusion channel, may be releasable from the chip to enable inspection of the cell cultures. The use of three-phase chambers allows for: low pressure (positive or negative relative to ambient) pumps to drive perfusion, and may use a 0.5-2 psi pressure difference to accomplish the same; tolerance to bubbles; and optionally, a very efficient bubble mixing of the medium. Metered removal of spent culture media and metered injection of fresh medium can be accomplished.

Accordingly, in one aspect, there is provided a perfuser comprising a patterned top surface and an opposing bottom surface, the top surface comprising: a plurality of receptacles, each receptacle comprising: a floor, an outlet, and at least one sidewall comprising a check microvalve comprising an inlet on the sidewall, and a plurality of supply channels each having a hydraulic diameter (d_{HC}) of 0.1-1 mm for (at least substantially) non-capillary transport of the liquid, each supply channel being fluidically connected to the inlet of one check microvalve, wherein a vertical separation between the inlet and the floor is at least 5 mm, wherein the plurality of receptacles comprises a first pair of receptacles and a second pair of receptacles, wherein the outlet of a first receptacle in each pair is configurable to be fluidically connected to a second receptacle of each pair.

In some embodiments, the check microvalve is a gravitationally checked microvalve (GCM), the GCM comprising an accretion surface on the sidewall at or near the inlet, wherein the accretion surface is adapted to guide formation and growth of a bolus of a liquid and limit horizontal spread of the liquid beyond the accretion surface, to draw a center of gravity of the bolus away from the first sidewall. In some embodiments, the outlet is located on the floor. In some embodiments, at least one of the outlets is fluidically connected to a vent at the bottom surface of the perfuser.

In some embodiments, the bottom surface of the perfuser is patterned to define at least part of a perfusion network that is fluidically connected to at least one of the outlets.

In some embodiments, the perfuser is composed of a material that comprises thermoplastic polymer.

In another aspect, there is provided a device for perfusing a cell culture comprising: a perfuser as described herein, a base layer having a top surface adapted to seal at least part of the bottom surface of the perfuser, wherein the base layer and the bottom surface of the perfuser together define a perfusion network that is fluidically connected to at least one of the outlets, a cover having a bottom surface adapted to seal at least part of the top surface of the perfuser, and a plurality of ports configured to couple to a pressure source, wherein each of the plurality of ports are fluidically connected to the plurality of receptacles, and at least one port is disposed at each opposite end of the device, wherein the perfusion network is configured to provide a first perfusion path and a second perfusion path between the at least one port at one end of the device and the at least one port at the opposite end of the device, wherein the perfusion network is configured such that the first pair of receptacles is fluidically interconnected through the first perfusion path, and the second pair of receptacles is fluidically interconnected through the second perfusion path, wherein the perfusion network comprises a central region, the central region having at least one central port open to the exterior of the device, wherein the perfusion network is configured to provide fluid communication between the central region and the first perfusion path at a first interface, and between the central region and the second perfusion path and a second interface, wherein the first and second interfaces are on opposite sides of the central region, and wherein the first interface is fluidically situated between the first pair of receptacles, and the second interface is fluidically situated between the second pair of receptacles.

In some embodiments, the perfusion network is configured such that the first and/or the second perfusion path is branched.

In some embodiments, the perfusion network is configured such that the first and/or the second perfusion path forms a loop.

In some embodiments, the bottom surface of the cover is patterned, and the top surface of the perfuser and the bottom surface of the cover together define an auxiliary network that is fluidically connected to the perfusion network.

In some embodiments, the perfusion network and/or auxiliary network comprises a metering receptacle and/or a micromixing receptacle.

In some embodiments, the perfuser and the base layer are discrete components. In others, the perfuser and the base layer are integrated as a single component.

In another aspect, there is provided a kit comprising a device as described herein and a pressure source.

In another aspect, there is provided a kit comprising a plurality of devices as described herein and a pressure source, wherein the plurality of devices are configured for multiplexed operation.

In another aspect, there is provided a method of operating a kit as described herein, comprising: a. fluidically connecting the pressure source to the ports of the device, b. providing a cell culture in the central region, and c. perfusing the cell culture, wherein the pressure source is operated at a relative pressure of no more than ±2 psi to perfuse a liquid medium along the first and second perfusion paths during step c.

In some embodiments, the first and second perfusion paths are independently controlled. In some embodiments, the first and second perfusion paths are reversed in direction at least once during step c, and in others, the first and second perfusion paths are provided in the same direction.

In some embodiments, the pressure source is operated at a relative pressure of about −0.05 to about −1 psi, preferably about −0.05 to about −0.5 psi.

A copy of the claims as filed and as granted are incorporated herein by reference.

Other aspects and features of the disclosure will become apparent to those ordinarily skilled in the art upon review of the following description of specific embodiments of the disclosure.

### BRIEF DESCRIPTION OF THE DRAWINGS

Embodiments of the disclosure will now be described in greater detail with reference to the accompanying drawings:

FIG. 1 illustrates: A: conceptual diagram of the three-lane design of a perfuser, wherein a central lane (c) for cell culture is flanked by two perfusion lanes (p) carrying media for perfusing the cell culture, that meet the central lane at respective interfaces (i); B: top plan, side elevation, and bottom plan views of one embodiment of a cell culture perfuser 100 having a three-lane design; and C: two oblique views of the perfuser 100 of FIG. 1B.

FIG. 2 illustrates the perfuser 100 of FIGS. 1B-1C; a base layer 112 comprising a microfluidic network and a bottom cover; and a top cover 111, the base layer and top cover adapted to seal the perfuser, which when assembled provide a cell culture perfusion device, A: one oblique view of the perfuser, base layer and top cover; B: another oblique view of the same;

FIG. 3 illustrates another embodiment of a cell culture perfuser having the same configuration as that of FIGS. 1 and 2, wherein the perfuser and microfluidic network are fabricated on the same layer or chip, A: top, elevation and bottom views; B: oblique views;

FIG. 4 illustrates the layer of FIG. 3, and bottom and top covers adapted to seal the layer, which when assembled provide a cell culture perfusion device, A: one oblique view of the layer and two covers; B: another oblique view of the same;

FIG. 5 illustrates another embodiment of a cell culture perfuser, wherein the perfuser and microfluidic network are fabricated on the same layer, where each perfusion lane is configured for recirculatory unidirectional flow along the interface region 215, A: top plan, side elevation, and bottom plan views of cell culture perfuser 200; B: two oblique views of the perfuser 200 of FIG. 5A;

FIG. 6 illustrates two embodiments of a pneumatically controlled cell culture perfusion device having a three-lane design, in a which a gel serving as the seed area for cell culture is suspended in the central lane, and perfused with media from the two flanking perfusion lanes controlled pneumatically via ports at either end of the device. A: Schematic of an embodiment where both perfusion lanes are controlled from the same ports, one at each end. B: Schematic of the embodiment also depicted in FIG. 3, where each perfusion lane is connected to a separate set of ports, allowing independent pneumatic control of both lanes. C: 3D rendering of the embodiment of FIG. 6A, wherein outlet 302 of first receptacle 301 is fluidically connected to outlet 304 of second receptacle 303. D: 3D rendering of the embodiment of FIG. 6B, with the central lane 305 marked in red.

FIG. 7 illustrates the embodiment depicted in FIG. 6A in operation, holding two different cell culture media in each perfusion lane, A hydrogel is suspended in the central lane. A: Media deposited into two receptacles on the right, one in each lane; B-F: negative pressure draws the media in both lanes to the left receptacles; G-J: media is drawn back to the original receptacles, from left to right; this sequence may be repeated until the media is replaced;

FIG. 8 illustrates another embodiment of a cell culture perfuser where each perfusion lane is connected to a separate set of ports, allowing independent pneumatic control of both lanes, A: 3D rendering of the perfuser, wherein outlet 402 of first receptacle 401 is fluidically connected to check microvalve 404 of second receptacle 403 where the check microvalve 404 enforces one-way flow of the perfusion lanes along the interface with the central region; and B: schematic of the perfuser;

FIG. 9 illustrates the embodiment depicted in FIG. 8 in operation, holding two different cell culture media in each perfusion lane, where a hydrogel is suspended in the central lane, A: media deposited into two receptacles on the right, one in each lane; B-G: negative pressure draws the media in the bottom lane to the bottom left receptacle; H-L: negative pressure draws the media in the top lane to the top left receptacle;

FIG. 10 conceptually illustrates the recirculatory unidirectional flow path of the embodiment of FIG. 5 where liquid in receptacles 205 is drawn past the interfaces 215 with the central lane into receptacles 206, and liquid from receptacles 206 is returned via receptacles 207 back to receptacles 205; and

FIG. 11 conceptually illustrates the recirculatory unidirectional flow path of the embodiment of FIG. 5 where liquid in receptacles 205 (marked in FIG. 11A) is drawn past the interfaces 215 with the central lane (location of interfaces marked in FIG. 11B) into receptacle 206 (marked in FIG. 11C), and liquid from receptacle 206 is returned via receptacle 207 (marked in FIG. 11D) back to receptacle 205.

### DETAILED DESCRIPTION

One or more embodiments are described to illustrate the invention by way of example. It will be appreciated that embodiments and examples are provided for illustrative purposes for teaching those skilled in the art, and are not meant to be limiting. All references to embodiments, examples, aspects, formulas, compounds, compositions, solutions, kits and the like is intended to be illustrative and non-limiting.

In one aspect, there is provided a perfuser comprising a patterned top surface and an opposing bottom surface, the top surface comprising: a plurality of receptacles, each receptacle comprising: a floor, an outlet, and at least one sidewall comprising a check microvalve comprising an inlet on the sidewall, and a plurality of supply channels each having a hydraulic diameter (d_{HC}) of 0.1-1 mm for non-capillary transport of the liquid, each supply channel being fluidically connected to the inlet of one checked microvalve, wherein a vertical separation between the outlet and the floor is at least 5 mm, wherein the plurality of receptacles comprises a first pair of receptacles and a second pair of receptacles, wherein the outlet of a first receptacle in each pair is configurable to be fluidically connected to a second receptacle of each pair.

As used herein, a “patterned surface” refers to a surface having a three-dimensional relief pattern. The patterned top surface of the perfuser defines at least parts of the supply channel and receptacle. The bottom layer of the perfuser and the any of the top and bottom surfaces of other layers (e.g., base layer and cover) disclosed herein may be patterned or not patterned. In some embodiments, any of the top and bottom surfaces of the perfuser and other layers (e.g., base layer and cover) disclosed herein, whether patterned or not, may further provide parts of vents, and other microfluidic channels, chambers, ports and vents, and possibly other three-phase receptacles and holds. As used herein, a “three-phase” microfluidic space is one in which a solid substrate supports a liquid, separated from a gas (e.g., air) by a free surface. These compartments are formed into or on the perfuser, which can be 3D-printed or otherwise fabricated as a single piece, or joined by over-molding. In some embodiments, the perfuser can further comprise, for example, other examples of the microfluidic check valve (i.e., check microvalve) described herein for feeding the same receptacle, or other three-phase holds, other valves, vias for microfluidic channels (pneumatic and/or liquid), and negative pressure sources or ports for coupling thereto. In some embodiments the perfuser is patterned on two opposing (i.e., top and bottom) surfaces, optionally with vias interconnecting the two sides, and the vias may have a higher d_{HC} value than segments of channels on the two sides.

In some embodiments, a ceiling of a receptacle may be provided by the bottom surface of the cover, which is optionally patterned. In some embodiments, at least a top side of the supply channel may be provided by the bottom surface of the cover, which is optionally patterned, such that the sidewall of the receptacle does not fully enclose the inlet. In some embodiments, certain compartments of the one or more networks (e.g., some or all receptacles and other three-phase structures) are formed on the perfuser, and other compartments (e.g., some or all two-phase structures) are formed on the cover.

In some embodiments, the outlet of the receptacle is located on the floor of the receptacle. In some embodiments, at least one of the outlets is fluidically connected to a vent at the bottom surface of the perfuser. In some embodiments, the bottom surface of the perfuser is patterned to define at least part of a perfusion network that is fluidically connected to at least one of the outlets.

In some embodiments, the perfuser is patterned, e.g., with segments of microfluidic channels, on both top and bottom surfaces that are adapted for sealed meeting with a cover and a base layer respectively.

The check microvalves provided herein can dose and dispense cell culture media in aliquots on the order of as low as 25 μL per dose. Precise control over the introduction of fresh media reduces cell shocking induced by the sudden replacement of large volumes of fresh media performed under conventional methods. The check microvalves can be operated using pressures below ambient pressure or low negative pressures to perform liquid manipulations, including valving. This expands the scope of materials suitable for and simplifies the design and manufacturing of the devices described herein and their seals. In some embodiments, the check microvalve is provided at an interface between a 2-phase and a 3-phase parts of a microfluidic network in a mesoscale device (i.e., chips having a height less than 2 cm, but more than 5 mm that are ideal for 3-phase microfluidics and too deep for typical, efficient, 2-phase microfluidics). Tailoring the valve around the valve opening allows for much smaller volume flows to reliably be check valved. The costs of deeper chambers for the 3-phase network segments, in terms of additional chip material, and forming times are quite small considering the efficiency and the reliability of the valving, and the low energy of the valving. Furthermore, very few materials come into contact with the liquid during valving, which reduces constraints on materials; and a “forgiving” microfluidic design is used, avoiding many issues with capillary flow and contact-angle limited (material) processes, avoiding reliability issues that plague other microvalves. In some embodiments, the check microvalve is tolerant of air bubbles entrained in the liquid, and permits microfluidic chip designs that avoid lost volumes used to avoid entrained bubbles.

In some embodiments, the check microvalve is a gravitationally checked microvalve (GCM). The GCM comprises an accretion surface on the sidewall at or near the inlet, wherein the accretion surface is adapted to guide formation and growth of a bolus of a liquid and limit horizontal spread of the liquid beyond the accretion surface, to draw a center of gravity of the bolus away from the first sidewall, thereby reducing a contact surface and adhesive force on the bolus.

A GCM, when compared with most pneumatically driven valves, requires only a slightly higher volume of chip material, and can advantageously be operated by low amplitude pressure, such as a pressure difference of less than 15 kPa (−2 Psi) (about 15% of ambient pressure on earth at sea level), and is preferably negative pressure (i.e., relative to ambience), without surface activation or hydrophilic or hydrophobic coatings in select areas. The technique provides for check valving of small volumes of liquid, such as aqueous liquids, hydrocarbons, and oils, with better control than would be expected of such a low-cost chip and low energy process.

Accordingly, a gravitationally checked microvalve (GCM) is provided for in a microfluidic device when resting on a horizontal surface. In operation, as the bolus grows while a surface contact area is constrained by the accretion surface, the bolus's weight eventually exceeds the adhesive force, and the bolus falls. In some embodiments, the volume of liquid accreted before fall is about 10 to about 500 μL.

One critical limit for the GCM therefore is a minimum amount of the liquid that must be delivered into the receptacle for check valving to occur, without risk of backflow of the liquid through the supply channel after the delivery. The minimum amount of liquid is associated with a critical bolus size, which is suspended above the floor for GCM valving. In some embodiments, a vertical separation between the outlet and the floor of the receptacle below the slide is about 5 mm or more. In some embodiments, the vertical separation is at least about 5.5 mm, at least about 6 mm, at least about 6.5 mm, at least about 7 mm, at least about 7.5 mm, at least about 8 mm, at least about 9 mm, or at least about 10 mm. In some embodiments, the vertical separation is in a range of about 5 to about 20 mm, about 5 to about 12 mm, about 5 to about 10 mm, about 6 to about 10 mm, about 7 to about 10 mm, about 8 to about 10 mm, about 9 to about 10 mm, about 5 to about 9 mm, about 6 to about 9 mm, about 7 to about 9 mm, about 8 to about 9 mm, about 5 to about 8 mm, about 6 to about 8 mm, about 7 to about 8 mm, about 5 to about 7 mm, about 6 to about 7 mm, or about 5 to about 6 mm. In some embodiments, the plenum of the supply channel is filled by the liquid, and the at least 5 mm vertical separation enables three-phase microfluidics within the receptacle.

The supply channel is fluidically connected to a check microvalve through which liquid leaves the supply channel into the receptacle. The supply channel may locally have an axis that is vertical, horizontal (i.e., perpendicular to the first sidewall), or at any angle therebetween. In some embodiments, the angle is lower than horizontal, as this may decrease splatter accumulating on the ceiling of the receptacle at an end of the delivery. In some embodiments, any one or more supply channels or any one or more other channels in the microfluidic network has a hydraulic diameter of about 0.1 mm to about 1 mm, about 0.2 mm to about 1 mm, about 0.3 mm to about 1 mm, about 0.4 mm to about 1 mm, about 0.5 mm to about 1 mm, about 0.6 mm to about 1 mm, about 0.7 mm to about 1 mm, about 0.8 mm to about 1 mm, about 0.9 mm to about 1 mm, 0.1 mm to about 0.9 mm, about 0.2 mm to about 0.9 mm, about 0.3 mm to about 0.9 mm, about 0.4 mm to about 0.9 mm, about 0.5 mm to about 0.9 mm, about 0.6 mm to about 0.9 mm, about 0.7 mm to about 0.9 mm, about 0.8 mm to about 0.9 mm, 0.1 mm to about 0.8 mm, about 0.2 mm to about 0.8 mm, about 0.3 mm to about 0.8 mm, about 0.4 mm to about 0.8 mm, about 0.5 mm to about 0.8 mm, about 0.6 mm to about 0.8 mm, about 0.7 mm to about 0.8 mm, 0.1 mm to about 0.7 mm, about 0.2 mm to about 0.7 mm, about 0.3 mm to about 0.7 mm, about 0.4 mm to about 0.7 mm, about 0.5 mm to about 0.7 mm, about 0.6 mm to about 0.7 mm, 0.1 mm to about 0.6 mm, about 0.2 mm to about 0.6 mm, about 0.3 mm to about 0.6 mm, about 0.4 mm to about 0.6 mm, about 0.5 mm to about 0.6 mm, 0.1 mm to about 0.5 mm, about 0.2 mm to about 0.5 mm, about 0.3 mm to about 0.5 mm, about 0.4 mm to about 0.5 mm, 0.1 mm to about 0.4 mm, about 0.2 mm to about 0.4 mm, about 0.3 mm to about 0.4 mm, 0.1 mm to about 0.2 mm, about 0.1 mm to about 0.3 mm, or about 0.1 mm to about 0.2 mm. In some embodiments, any one or more supply channel or any one or more other channel in the microfluidic network has a hydraulic diameter of about 0.1 mm, about 0.2 mm, about 0.3 mm, about 0.4 mm, about 0.5 mm, about 0.6 mm, about 0.7 mm, about 0.8 mm, about 0.9 mm, or about 1 mm. The supply channel may comprise any number of segments, with different directions, along its length, and in some embodiments the supply channel includes at least one via segment that couples patterning on opposite sides of the perfuser, and in some embodiments the two patterned sides of the substrate. Preferably one of the two patterned sides meets a bottom cover that defines the floor, or is within 10% of a thickness of the chip from the depth of the floor, and the other provides a top meeting surface for a top cover. In some embodiments, the supply channel includes at least one serpentine channel segment (horizontally or vertically oriented) for slowing liquid flow through (i.e., increasing fluidic resistance of) the supply channel. In some embodiments, two or more supply channels are fluidically connected to the same inlet of one receptacle. In some embodiments, if, for forming reasons, minimum dimensions of the supply channel are greater than the desired d_{HC}, meets of the segments can have reduced d_{HC} (such as via segments formed by larger diameter pins) than desired for the supply channel.

The liquid may be supplied to the supply channel by any suitable means. In some embodiments, the liquid is supplied through the inlet by action of positive relative pressure (to ambient, or to the pressure supplied at the opposite end). In some embodiments, the liquid is supplied through the inlet by action of negative pressure. In some embodiments, the perfuser further comprises a supply chamber fluidically connected to the supply channel for containing the aqueous liquid upstream of the supply channel. In some embodiments, the supply chamber is vented. In some embodiments, the supply channel extends between an outlet at or near a floor of the vented supply chamber to the inlet at or near a top of the receptacle. The floor of the supply chamber and receptacle preferably differ by less than 10% of the perfuser's thickness.

The receptacle has a floor and at least the first sidewall. In some embodiments, the receptacle defined by the substrate has an open top. In some embodiments, the receptacle will have a ceiling, or the perfuser will be covered to provide the ceiling. In some embodiments the ceiling comprises a lower surface of a cover that covers the perfuser. In some embodiments, the perfuser comprises a plurality of the microfluidic check valves and respective supply channels for feeding a plurality of liquids into a receptacle, which is common to the plurality of the microfluidic check valves. In some embodiments, two or more of the plurality of microfluidic check valves open into the receptacle on the same sidewall. In some embodiments, two or more of the plurality of microfluidic check valves open into the receptacle on different sidewalls. In some embodiments, the perfuser comprises a plurality of receptacles with respective GCMs. In some embodiments, the receptacles are interconnected by microfluidic channels.

In some embodiments, one or more of the receptacles are fluidically connected to a port configured to couple to a pressure source. In some embodiments, a perfuser, or a device comprising the perfuser, comprises a plurality of ports configured to couple to a pressure source, wherein each of the plurality of ports are fluidically connected to the plurality of receptacles, and at least one port is disposed at each opposite end of the perfuser or the device. In some embodiments, the plurality of ports are co-operable with a pressure source to apply negative relative pressure of no more than ±2 psi (more preferably −0.03 to −0.7 psi) to control fluid displacement through the perfuser or the device, e.g., to perfuse a liquid medium along the first and second perfusion paths. In some embodiments, either or both perfusion paths fluidically interconnect a plurality of receptacles, e.g., two, three, four, five, six, seven, or eight receptacles, each receptacle being fluidically connected to a corresponding port. In some embodiments, either or both perfusion paths fluidically interconnect a plurality of receptacles, e.g., two, three, four, five, six, seven, or eight receptacles in a loop which may be unidirectional (i.e., allowing one-way flow only) or bidirectional (allowing two-way flow), depending on the configuration of check microvalves within the perfusion path. In some embodiments, the perfuser or the device comprises 4, 5, 6, 7, 8, 9, 10, 11, 12, 13, 14, 15, or 16 receptacles. In some embodiments, the perfuser or the device comprises 4-10, 4-8 or 4-6 receptacles. In some embodiments, the perfuser or the device comprises 2, 3, 4, 5, 6, 7, 8, 9, 10, 11, 12, 13, 14, 15, or 16 ports. In some embodiments, the perfuser or the device comprises 2-10, 2-8, 2-6 or 2-4 ports.

In some embodiments, one or more of the receptacles has a depth-to-width aspect ratio of less than 2:1, preferably less than 1:1, more preferably less than 0.5:1. In some embodiments, one or more of the receptacles have a mean depth of at least about 5 mm, at least about 5.5 mm, at least about 6 mm, at least about 6.5 mm, at least about 7 mm, at least about 7.5 mm, at least about 8 mm, at least about 9 mm, or at least about 10 mm. In some embodiments, one or more of the receptacles have a mean depth of no more than about 20 mm, no more than about 19 mm, no more than about 18 mm, no more than about 17 mm, no more than about 16 mm, or no more than about 15 mm. In some embodiments, a receptacle may define a volume that is cuboid, prismatic, cylindrical, discoid, conical, or spheroid, or in a shape of a frustum of any of the foregoing shapes. When the check microvalve is a GCM, the receptacle dimensions preferably accommodate the critical volume of the bolus so that the inlet is separated from a free surface of the receptacle after fall by at least two times a radius of the bolus of threshold weight and volume, preventing back flow from the receptacle through the supply channel. The GCM works in conjunction with an enclosure upstream and/or downstream of the supply channel, adapted to enable a pressure difference on opposite sides of the enclosure to drive liquid through the supply channel to the inlet of the receptacle. In some embodiments, the enclosure may be provided only upstream of the GCM, for example if positive pressure is used to push the liquid into the microvalve. In some embodiments, negative pressure is used for pulling the liquid, which may improve control. In some embodiments, the enclosure includes at least part of the supply channel, and extends over the receptacle, in at least one state of operation of the device, and that a vent is provided upstream of the supply channel.

The GCM described herein locally resembles a waterfall and may be formed as a component of a perfuser that may be manufactured as a single layer. While the structure may superficially look like a waterfall, with special attention paid to a front surface thereof, it in no way resembles a waterfall in operation.

To effect valving, the enclosure needs a seal that permits the receptacle to have a lower pneumatic pressure than the supply chamber, for a duration sufficient to overcome inertia and fluid resistance in moving from the supply chamber into the receptacle, and to grow the liquid on the accretion surface. Capillary effects are second-order in the operation of the GCM.

The cover, more specifically the bottom surface thereof, may provide a ceiling of the receptacle. If so, the inlet on the sidewall of the receptacle is preferably: sufficiently lower than the ceiling to ensure that the bolus does not contact the ceiling during accretion; or defined in part by the ceiling, so that the ceiling takes part in forming the supply channel, at least at the inlet. Various forming routes make forming channels at interfaces of substrates and covers quite efficient. An undemarcated portion of the ceiling may be implicated in the accretion process, such that the bolus, during accretion, will spread over the ceiling. In general, the constraints provided by the accretion surface act to limit spread in the horizontal directions over the sidewall, and these constraints discourage spreading over the ceiling in the same vector directions, but in contrast nothing limits spreading in the perpendicular direction, which is generally towards the centre of the receptacle. Thus, there is a tendency for the liquid to spread towards the centre of the receptacle, which further increases the draw of the liquid away from the first sidewall. As the horizontal spread limits constrain flow in two directions, the bolus is prompted to grow in the others, which pulls the bolus towards the centre of the receptacle, as well as vertically up (or horizontally away from the wall) and down. So while the increased surface area of contact, provided by the ceiling, does increase adhesion of the bolus (which in turn requires a greater mass before fall), and also undesirably implicates an edge in the contact surface area, the strong limit on vertically upward movement, and pull that is directly away from a periphery of the top edge of the receptacle, ultimately increases a distance of the centre of mass of the bolus from the first sidewall, which expedites shedding (i.e., fall) of the bolus. Furthermore, locating the inlet at the interface between the cover and the perfuser may be preferable for low-cost chip forming and assembly.

The GCM is a one-way valve between a two-phase supply channel and a three-phase receptacle into which the liquid from the supply channel is being conditionally delivered, and not returnable after delivery. The valve is formed into the perfuser at the mouth of the supply channel, and is surrounded, at least on 3 sides, by an accretion surface that receives the aqueous liquid from the supply channel during accretion. The liquid grows to form a bolus on the accretion surface, while the pressure difference aliments the bolus. The bolus grows within an accretion volume bounded in part by the accretion surface. By limiting the spread of the bolus across the sidewall, the bolus is forced to bulge outwardly, which expedites fall of the bolus, principally by decreasing the surface area of contact, which both decreases a threshold bolus size required to overcome attraction to the accretion surfaces by decreasing the adhesion, and by increasing a volume suspended, which forces a centre of mass of the bolus to extend further away from sidewall and the outlet.

The accretion surface is a single (i.e. connected) surface of the sidewall that has a function of prescribing and limiting a direction of growth of a liquid bolus at the mouth of the supply channel, in terms of directions in which the growth is encouraged or discouraged. While various methods of guiding surface movement of liquid are known in the art, these typically require: fine scale surface patterning, selective surface activation, or microfluidic inserts, all of which are possible, but these each have disadvantages in terms of speed and cost of production, and reliability-in particular reliability after long-term storage in low-cost storage conditions. In some embodiments, substantially large-scale structural features in the form of one or more (preferably two) limit edges provide delimitation of and by the accretion surface. A limit edge is a drop-off or recess formed by a second surface that adjoins the accretion surface at the limit edge. The limit edge reduces propensity of liquid to spread from the accretion surface to the second surface. As such the angle between the accretion surface and second surface should have an internal (i.e., measured within the sidewall, rather than in the void space of the receptacle) angle of less than 160°, preferably less than 145°. In some embodiments, the internal angle is from about 90°to about 145°. In some embodiments, the internal angle is reflex, e.g., about 225°; an edge so formed does not provide a growth-limiting effect. It will be appreciated that no forming methods produce mathematically ideal structures, and edges are often the least perfectly formed features. For present purposes, the sharper the limit edges, the shorter the distance the accretion surface needs to be projected from the sidewall, and the less the material that is needed to project the accretion surface into the receptacle. Invariably, limit edges have some measure of rounding that can be specified by a nominal radius of curvature. The smaller the radius, the sharper the limit edge. To limit spread, the limit edge preferably has a radius of curvature smaller than ¼ the projection distance (i.e., the distance the limit edge is projected into the receptacle from the sidewall). Thus, the accretion surface guides growth and formation of the bolus, by permitting the liquid from the supply channel to spread over the accretion surface along its full (but limited) extent in a horizontal direction, without texturing, or selectively coating, sidewall parts. In some embodiments, limit edges are provided on both sides of the inlet, to pin the bolus to the accretion surface and limit spread in either horizontal direction, enabling the pinching of the bolus.

Any limit edges will tend to increase a bulge of the bolus, and therefore should increase propensity for the bolus to fall with lower volume. In some embodiments, the limit edge is provided a distance from the inlet (such as at least 3 μm, and more preferably at least 10 μm) to permit an initial bolus to grow to a certain size before any physical limit is encountered (hence the accretion surface surrounds at least three sides of the inlet). That said, the greater a distance of the limit edge from the outlet, the larger a threshold bolus size (i.e., bolus size for which weight overcomes adhesion) will be. Barely any pinching effect is possible for most aqueous liquids if the limit edge is 4-5 mm away from the outlet. As such, the limit edge, in some embodiments, is less than 2.5 mm, and more preferably less than 0.75 mm, away from a centre of the outlet, to allow bolus growth until it reaches the limit edges. The limit edges run vertically, or substantially vertically, and limit horizontal spread of the bolus.

The accretion surface can be flat, essentially planar, simply curved or bi-curved, e.g., concave to decrease the internal angle of the limit edges. Any curvature of the accretion surface is preferably shallow such that a radius of curvature at any point on the surface is greater than a peripheral length of the inlet, which is preferred for avoiding any liquid being adhered to the accretion surface after fall of the bolus. Regardless of such slight curvature, the accretion surface has a mean orientation defined by a normal. The normal may be oriented horizontally, and if so, the accretion surface is a vertical face of the first sidewall, or projected from the first sidewall by the ridge. Herein a 0.25-5°draft angle commonly used to facilitate demolding in some forming processes is considered vertical. In some embodiments, the normal of the accretion surface has a negative inclination. A negative angle, in this context, refers to an orientation for which a ray coincident to the normal meets a horizontal plane of the floor, presumably at a fairly acute or glancing angle. In other words, the normal is directed at an angle below horizontal. While verticality or a negative angle is desired, and can typically be readily formed, in some embodiments it is preferable to use a positive angle, whereby the normal is somewhat above horizontal. In respective embodiments the positive angle is no more than: 45°, 40°, or 30°.

In some embodiments, the two limit edges are formed by a ridge, defined by the accretion surface and flanking second surfaces, that projects the inlet into the receptacle. The ridge may be shaped, in horizontal cross-section, as an acute trapezoid (trapezoid with acute angles at long base), oriented with the long base meeting the first sidewall. The acute trapezoid may be symmetric (isosceles), to provide equal resistance to flow at both sides.

In some embodiments, the ridge extends towards the floor only as far as the accretion surface, and in these embodiments, the bottom edge of the accretion surface adjoins a slide that extends to the floor of the receptacle. In some embodiments, the receptacle is narrowed below the accretion surface to avoid edges of the walls meeting the floor of the receptacle, and the attendant risks of dead volumes associated therewith. The ridge can taper gradually or abruptly below the accretion surface. Alternatively, the accretion surface may be an undifferentiated part of the same ridge face (e.g., short base surface if trapezoid) that provides the slide from the accretion surface to the floor.

The slide is oriented within 30°of vertical when the perfuser is resting on a horizontal surface. In some embodiments, the slide is curved. The slide extends from the accretion surface to the floor of the receptacle. When the bolus accreting on the accretion surface grows heavy enough to overcome the adhesion holding the bolus, the bolus detaches and falls down the slide to the bottom of the receptacle. In some embodiments, the slide extends all the way to the floor of the receptacle. In some embodiments, the slide extends to about a fill level for the liquid in the receptacle. In some embodiments, the slide is 1-3 mm wide, on average. In some embodiments, the accretion surface comprises a vertically oriented trench in the protrusion for guiding the bolus to follow the slide, and the trench may be a V-shaped groove that narrows progressively towards the floor, with the V-shape having an angle of 20°-150°.

In some embodiments, the inlet comprises a nozzle for changing a velocity profile of liquid passing therethrough, which can be particularly helpful as a final portion of the liquid in the supply channel enters the receptacle through the inlet. The nozzle may constrict or expand the inlet in either or both directions (i.e., width and depth).

In some embodiments, the accretion surface surrounds the outlet completely and the inlet is placed at a distance (e.g., about 4 mm) below the top of the receptacle, to avoid interaction of the bolus with a ceiling of the receptacle. In some embodiments, the inlet is positioned at the top of the sidewall such that the ceiling of the receptacle, if present (e.g., provided by a cover) defines part of the accretion surface.

In some embodiments, the portion of the accretion surface defined by the ceiling is unmodified relative to the rest of the surface of the receptacle. In some embodiments, the portion of the accretion surface defined by the ceiling is patterned or otherwise modified. While the ceiling allows the bolus to spread during accretion, and increases surface adhesion of the bolus, it ultimately increases a distance of the centre of mass of the bolus from the first sidewall, in concert with the pinching effect of the limit edges. Because the limit edges constrain the bolus from spreading horizontally along the first sidewall and the ceiling precludes spreading upwardly, the bolus spreads freely across the ceiling towards a centre of the receptacle, and downwardly, but far less in the directions of the sidewall than it would absent the limit edges.

In some embodiments the shape of the accretion area encourages bolus growth that tends to draw liquid away from the first sidewall. The native contact angle of materials typically used to form the receptacle and ceiling will allow for bolus formation in a fairly robust manner. The pinching provided by the two limit edges, and spread across the ceiling, both draw the bolus away from the accretion surface. When the bolus has accreted enough liquid so that the weight of the bolus exceeds the adhesion, the bolus falls.

In another aspect, there is provided a device for perfusing a cell culture comprising: a perfuser as described herein, a base layer having a top surface adapted to seal at least part of the bottom surface of the perfuser, wherein the base layer and the bottom surface of the perfuser together define a perfusion network that is fluidically connected to at least one of the outlets, a cover having a bottom surface adapted to seal at least part of the top surface of the perfuser, and a plurality of ports configured to couple to a negative pressure source, wherein each of the plurality of ports are fluidically connected to the plurality of receptacles, and at least one port is disposed at each opposite end of the device, wherein the perfusion network is configured to provide a first perfusion path and a second perfusion path between the at least one port at one end of the device and the at least one port at the opposite end of the device, wherein the perfusion network is configured such that the first pair of receptacles is fluidically connected through the first perfusion path, and the second pair of receptacles is fluidically connected through the second perfusion path, wherein the perfusion network comprises a central region, the central region having at least one central port open to the exterior of the device, wherein the perfusion network is configured to provide fluid communication between the central region and the first perfusion path at a first interface, and between the central region and the second perfusion path and a second interface, wherein the first and second interfaces are on opposite sides of the central region, and wherein the first interface is fluidically situated between the first pair of receptacles, and the second interface is fluidically situated between the second pair of receptacles.

A microfluidic device as described herein may have one or more of the following advantages: simpler in design; requires no surface activations; is amenable to large scale manufacturing; permits reliable checked valving; and exhibit high resistance to backflow, with very little energy or control equipment. In operation, the GCM is effective at eliminating bubbles entrained in the supplied liquid, and can offer very low dead volume losses.

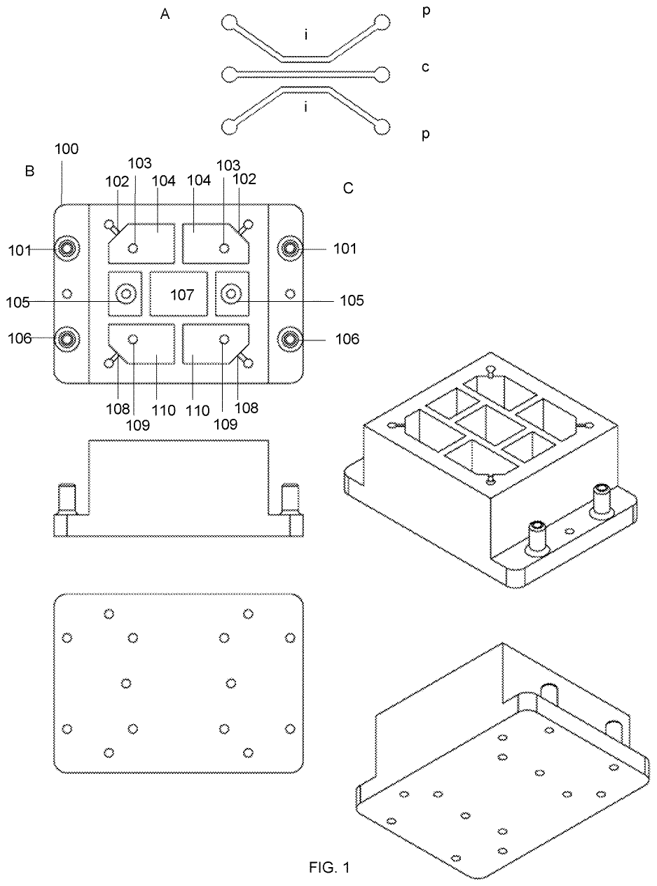

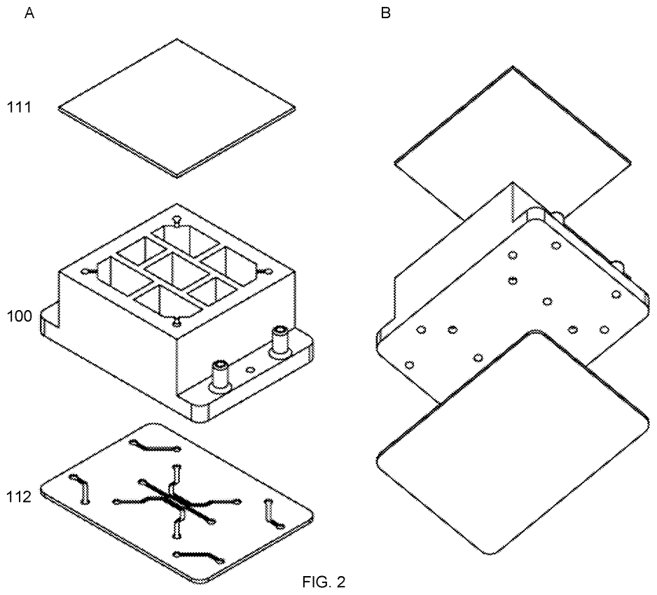

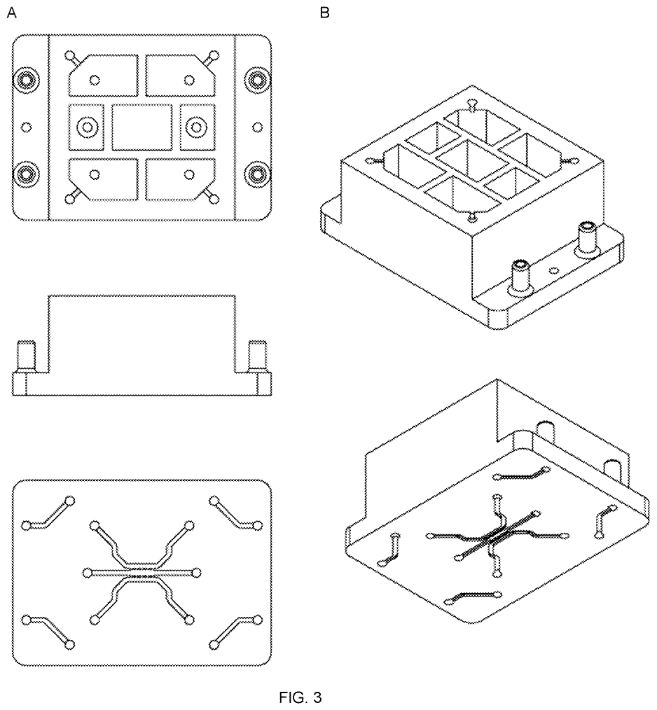

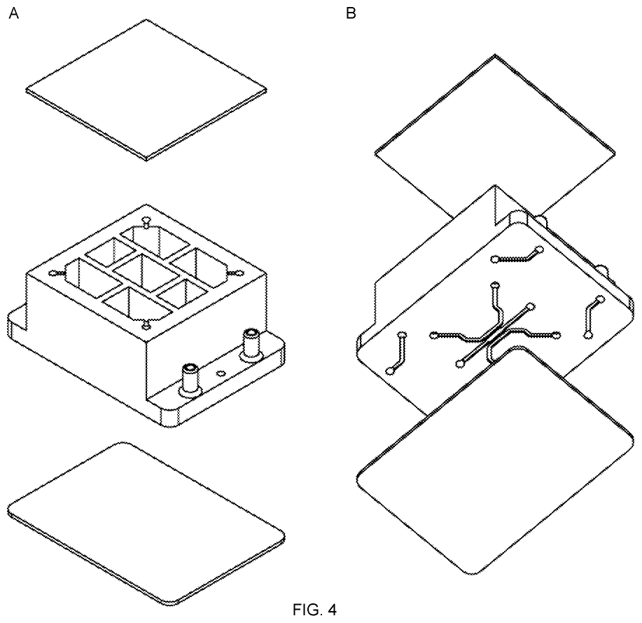

FIG. 1B shows one embodiment of the perfuser 100 as described herein having a three-lane design, in which each perfusion lane may be independently controlled. A central channel interconnects a pair of central ports 105 open to the top face of the perfuser, through which a gel and/or cells may be introduced for culture. One perfusion lane is defined between a pair of ports 101 which are configured to couple to a pressure source. Each port 101 is fluidically connected to a respective receptacle 104 at check microvalve 102 via a supply channel (not shown). A channel (not shown) connects each outlet 103 of each receptacle 104 with an interface (not shown) with the central channel located within viewing window 107. Similarly, another perfusion lane is defined between a pair of ports 106 which are configured to couple to a pressure source. Each port 106 is fluidically connected to a respective receptacle 110 at check microvalve 108 via a supply channel (not shown). A channel (not shown) connects each outlet 109 of each receptacle 110 with an interface (not shown) with the central channel located within viewing window 107. Grooves for the channels may be patterned on a separate base layer 112 (FIG. 2), or they may be patterned on the bottom side of the perfuser (FIGS. 3 and 4). The perfuser 100 may be sealed with the base layer 112 and top cover 111 to enclose the microfluidic network, providing a cell culture perfusion device that can be pneumatically operated with a pressure source connected to ports 101 to push liquid through the top perfusion lane, and to ports 106 to independently push liquid through the bottom perfusion lane.

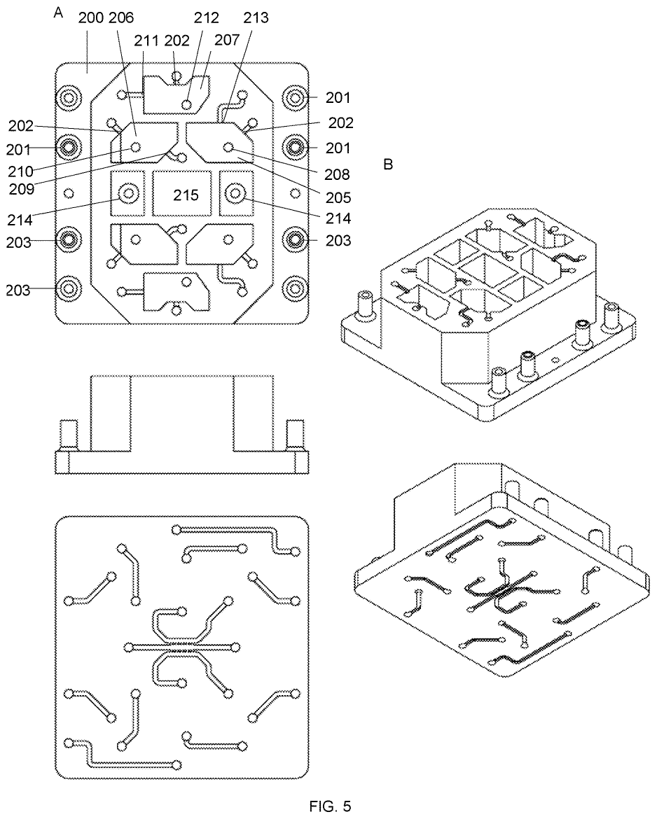

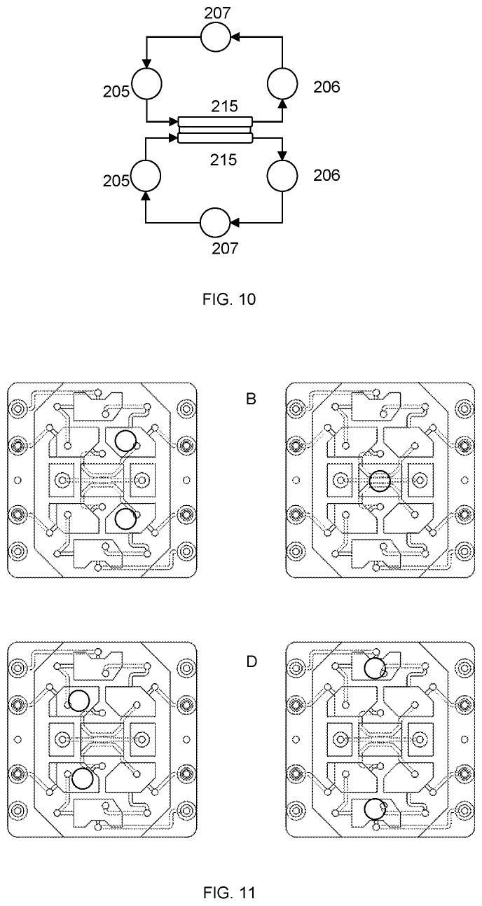

FIG. 5 shows another embodiment of a perfuser 200 also having a three-lane design, wherein the two perfusion paths are configured for unidirectional recirculatory flow along the interface with the central channel located within viewing window 215. As shown in FIGS. 10 and 11 (see Brief Description of the Drawings), each perfusion lane connects receptacles 205, 206 and 207 in a one-way loop, with the interface with the central channel being located between receptacles 205 and 206. Each independently operable port 201 is fluidically connected to a respective receptacle 205, 206 and 207 at check microvalve 202 via a supply channel (not shown). In the top perfusion lane, a first channel (not shown) connects outlet 208 of receptacle 205 via an interface (not shown) with the central channel located within viewing window 215, and to receptacle 206 at check microvalve 209. A second channel (not shown) connects outlet 210 of receptacle 206 to receptacle 207 at check microvalve 211. A third channel connects outlet 212 of receptacle 207 to receptacle 205 at check microvalve 213, closing the loop. The bottom perfusion lane also contains three receptacles similarly configured in a loop, with each receptacle being independently fluidically connected to one of the ports 203.

In some embodiments, the device further comprises additional layers for different functions. In some embodiments, the device further comprises one or more inserts between the perfuser and either or both of the base layer and cover, for example luer locks, readout zones, and inspection areas.

The device as a whole, or the perfuser or any of the other layers (e.g., base layer or cover) described herein on their own, may define one or more microfluidic networks, such as a perfusion network or an auxiliary network, that comprise one or more check microvalves (e.g., GCMs), receptacles, ports, vents, channels, and other microfluidic features. The one or more microfluidic networks may comprise channels that interconnect various chambers and/or open externally to an external environment, tubing to other supplies, or couplers to devices like loading pins, micropipettes, or syringes. In some embodiments, the channels comprise a plurality of microfluidic channels have d_{HC} values within 10% of that of the supply channel. Each channel may interconnect at least one chamber or receptacle, to another chamber or receptacle, or to a port of the chip. In some embodiments, each port provides one or more of the following functions: a liquid supply or extraction port; a pressurized gas supply or extraction port; or a passive vent, open to ambience. In some embodiments, the same port can be used for two or more of these three uses at different time points in operation. In some embodiments, a majority of the microfluidic channels are defined by segmented pieces, including at least one segment that is a via that extends through the perfuser or other layer to connect two patterned surfaces on opposite sides of the perfuser or other layer.

In some embodiments, the base layer is patterned to define (with the unpatterned bottom surface of the perfuser) a perfusion network comprising microfluidic channels, optionally excluding vias. In some embodiments, the perfuser and cover are bonded together, or adapted to bond together, to partially or fully enclose one or more microfluidic networks, such as a perfusion or auxiliary network. In some embodiments, the perfuser, the base layer and/or the cover are discrete components, which may be reversibly attached to and detached from one another. In some embodiments, the perfuser is integrated with the base and/or cover as a single component, e.g., they are manufactured as a single chip. In some embodiments, the perfuser and the base layer are integrated as a single component.

To increase the number of fluidic operations on liquids in the receptacle, in some embodiments, the receptacle is fully enclosed, the enclosure being defined with a seal between the top surface of the perfuser and the bottom surface of the cover of the device. In some embodiments, one or more ports of the device are provided for coupling to a positive or negative pressure source to move liquid through the valve, or to mix the liquid in the receptacle, or to remove the liquid from the receptacle, for example. In some embodiments, the enclosure covers the receptacle and a pressure port in fluid communication with the receptacle, the pressure port fluidically couplable, or coupled, to a source of negative pressure (relative to ambient pressure). In some embodiments, applying negative pressure to the pressure port moves the liquid through the valve into the receptacle, permits bubble mixing of liquid in the receptacle, moves the liquid out of the receptacle, or any combination thereof.

The perfuser and other layers described herein may be composed of any suitable material. In some embodiments, the material is or comprises a metal, or a polymeric material, and it may be natively hydrophobic and remain untreated. In some embodiments, the material is or comprises a thermoset, or a thermoplastic polymer, such as a thermoplastic elastomer. In some embodiments, the material is or comprises a thermoplastic polymer. In some embodiments, the material is or comprises silicone. The material may be chosen for biocompatibility, reasonably low reactivity with subject liquids, and is preferably not gas-permeable. The wide variety of polymers useful for microfluidics are well known. There is a particular use for glass and transparent plastic at least for covers, to facilitate viewing of liquid during displacements, and in some cases for readout and inspection. The wide variety of polymers useful for microfluidics are well known. In some embodiments, the thermoplastic polymer is polycarbonate or cyclic olefin polymer (e.g., ZEONOR®). In some embodiments, the perfuser or any of the other layers of the device, such as the base layer or cover, is made by injection molding or 3D printing. In some embodiments, the perfuser or any of the other layers of the device, such as the base layer or cover, is provided with an adhesive to bond and seal two layers.

In some embodiments, the perfusion network is configured such that the first and/or the second perfusion path is branched. Branching allows for the incorporation of additional microfluidic compartments and networks for auxiliary functions on the device as described below. In some embodiments, the perfusion network is configured such that the first and/or the second perfusion path forms a loop, so that recirculating flow through the device can be achieved.

In some embodiments, the bottom surface of the cover is patterned, and the top surface of the perfuser and the bottom surface of the cover together define an auxiliary network that is fluidically connected to the perfusion network. The auxiliary network may provide two-phase or three-phase microfluidic channels, chambers and receptacles for auxiliary functions potentially requiring precisely metered volume control such as injecting fresh media, withdrawing spent media, testing and sampling cell media, reporting accumulated target species, and injecting drugs, adjuvants, growth modifiers, or other solids, liquids, or gases such as oxygen or carbon dioxide.

In some embodiments, the perfusion network and/or auxiliary network comprises a metering receptacle and/or a micromixing receptacle. One or more receptacles of the device may incorporate metering capabilities. Consistent metering of spent culture media allows cells to avoid shocks, e.g., unwanted spikes in glucose levels, every time fresh media is introduced. Retaining a metered portion of spent media whenever fresh media is introduced may be advantageous to cell growth. Consistent low-volume metering also allows for the removal of small aliquots of spent media for off-chip analysis. One or more receptacles of the device may incorporate micromixing capabilities. Bubble mixing allows distribution of dissolved oxygen and carbon dioxide at levels that are more physiologically relevant than those provided by traditional gas diffusion-based methods. Such metering and micromixing capabilities may be particularly useful for controlling liquid flows in the multiplex operation of these devices.

In another aspect, there is provided a kit comprising a device as described herein and a pressure source. The pressure source may be a source of positive pressure, negative pressure, or both positive and negative pressure. In some embodiments, the pressure source provides both positive and negative pressure. In some embodiments, the pressure source is a positive pressure source. In some embodiments, the pressure source is a negative pressure source. In some embodiments, the pressure source is a pump, such as a micro diaphragm pneumatic pump. In some embodiments, the pressure source is low-power (e.g., 2 to 20 mW) and portable. In some embodiments, the pressure source is coupled to the plurality of ports using tubing, such as laboratory-grade silicone tubing. In some embodiments, the tubing is outfitted with filters such as HEPA filters to prevent contamination of the off-device elements of the system.

In some embodiments, the pressure source is operable or operated at a relative pressure of no more than about ±1.5 psi, no more than about ±1.0 psi, or no more than about ±0.5 psi. Operating the devices and kits described herein at these lower pressures decreases requires bonding strength requirements and relaxes the need for strong seals to prevent leakage during operation.

In some embodiments, the negative pressure source is operable or operated at a relative pressure of about −0.05 to about −2 psi, −0.05 to about −1.5 psi, −0.05 to about −1 psi, about−0.05 to about −0.95 psi, about −0.05 to about −0.9 psi, about −0.05 to about −0.85 psi, about −0.05 to about −0.8 psi, about −0.05 to about −0.75 psi, about −0.05 to about −0.65 psi, about −0.05 to about −0.6 psi, about −0.05 to about −0.55 psi, or about −0.05 to about −0.5 psi. In some embodiments, the negative pressure source is operable or operated at a relative pressure of about −0.05 to about −0.5 psi.

In some embodiments, the positive pressure source is operable or operated at a relative pressure of about 0.05 to about 2 psi, about 0.05 to about 1.5 psi, about 0.05 to about 1 psi, about 0.05 to about 0.95 psi, about 0.05 to about 0.9 psi, about 0.05 to about 0.85 psi, about 0.05 to about 0.8 psi, about 0.05 to about 0.75 psi, about 0.05 to about 0.65 psi, about 0.05 to about 0.6 psi, about 0.05 to about 0.55 psi, or about 0.05 to about 0.5 psi. In some embodiments, the negative pressure source is operable or operated at a relative pressure of about 0.05 to about 0.5 psi.

In some embodiments, the kit comprises a plurality of devices as described herein, wherein the plurality of devices are configured for multiplex operation. As will be readily understood by a person skilled in the art, multiple copies of the devices as described herein may be fabricated on a microplate with the receptacles arranged in a fashion similar to traditional wells on multiwell plates. For instance, 24, 36, 48 or 96 of the devices may be incorporated on a single plate depending on the volume of cell culture media required in each device. A person skilled in the art will appreciate that any number of devices on such a plate may be connected together for multiplex uni- or bidirectional two-lane perfusion cell culturing, wherein each perfusion lane may be individually controlled. The precision of control over media flow provided by multiplex operation of the disclosed pneumatic devices is advantageous compared to current multiplex systems based on gravity-driven hydrostatic flow. Multiplex operation of the devices disclosed herein may be compatible with automated liquid handling systems for high-throughput drug screening.

In another aspect, there is provided a method of operating a kit as described herein, comprising: a. fluidically connecting the pressure source to the ports of the device, providing a cell culture in the central region, and perfusing the cell culture, wherein the pressure source is operated at a relative pressure of no more than ±2 psi to perfuse a liquid medium along the first and second perfusion paths during step c.

Positive and negative pressures may be used to simulate different physiological processes. For example, negative pressure may be used to simulate blood flow dynamics and their effects on cells. Pneumatic control of the devices presently disclosed allows physiological fluid flow to be mimicked more closely than gravity-based flows, enabling more realistic simulation of inter-cell, inter-tissue, or inter-organ communication in cultures.

In some embodiments, the cell culture perfusion devices disclosed herein have a three-lane design (FIG. 1A), in a which a gel serving as the seed area for cell culture is suspended in the central lane or region, which is perfused with liquid (e.g., cell culture media) from two perfusion lanes controlled pneumatically via ports at either end of the device. The two perfusion lanes define a first and second perfusion path for the liquid to flow through the network from one side of the device to the other. The perfusion network is configured to provide fluid communication between the central region and the first perfusion path at a first interface, and between the central region and the second perfusion path and a second interface. The first and second interfaces are on opposite sides of the central region; in other words, the first and second perfusion lanes flank the central lane or region. The first interface is fluidically situated between the first pair of receptacles, and the second interface is fluidically situated between the second pair of receptacles. This allows fluid flow at the interface of each perfusion path to be controlled by pneumatics.

In some embodiments, the perfusion paths are controlled from the same ports, one at each end. In some embodiments, the first and second perfusion paths are independently controlled, wherein each perfusion lane is connected to a separate set of ports to which different pressures can be applied, in some embodiments simultaneously.

In some embodiments, the first and second perfusion paths are reversed in direction at least once during perfusion. This allows the simulation of reversing or oscillating flows.

In some embodiments, the first and second perfusion paths are provided in the same direction. Consistent unidirectional flow is desired in many perfusion applications. The perfusion network may be configured such that the first and/or the second perfusion path forms a loop, enabling media flow to be maintained continuously in the same direction over the cell culture during perfusion. In some embodiments, the first and second perfusion paths are provided in opposing directions.

The present disclosure will be further illustrated in the following examples.

### Example 1: Demonstration of Perfusion Flow in a Cell Culture Microfluidic Device With a Three-Lane Design

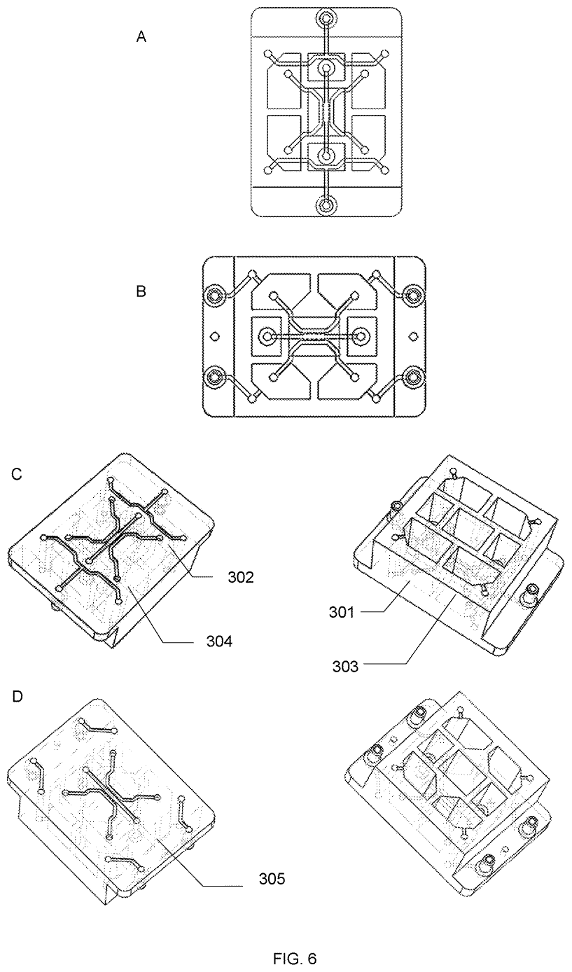

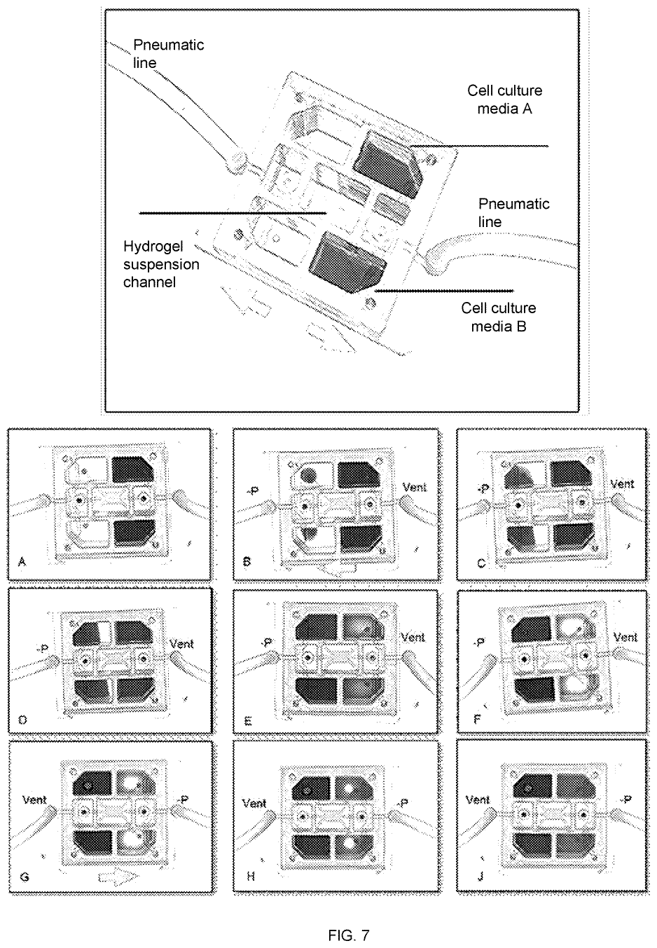

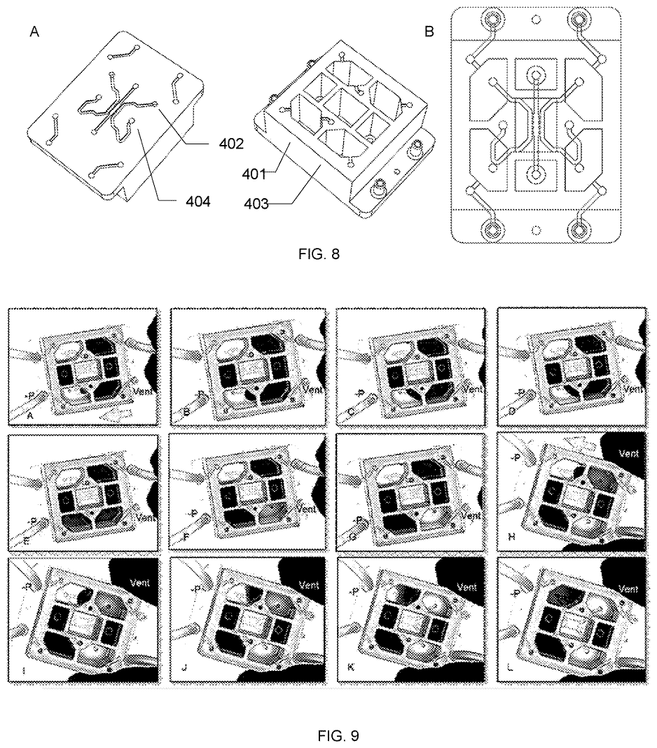

FIG. 7 illustrates perfusion flow in a cell culture microfluidic device with a three-lane design. The central lane is open to the top surface of the device at two ports, one at each end of the lane, through which hydrogel and cells may be introduced. FIG. 7 depicts a hydrogel suspended in the central lane. Flanking the central lane are two perfusion lanes connected to the ports on either side of the device. Each perfusion lane connects two three-phase receptacles with an interface with the central lane at the middle window. The ports are connected to pneumatic lines. A: Cell culture media is deposited into the two receptacles on the right, one in each perfusion lane. A cover is applied to seal the top side of the device, including the receptacles and the central lane ports. B-F: Negative pressure is applied to the left pneumatic line, while the right pneumatic line serves as a vent. This results in the media in both lanes being drawn from the right to the left receptacles. G-J: The configuration of the pneumatic lines is reversed: negative pressure is applied to the right pneumatic line, while the left pneumatic line serves as a vent. Accordingly, media is drawn back to the receptacles on the right. This sequence may be repeated as needed to simulate oscillatory flow.

### Example 2: Demonstration of Independent Control of Two Perfusion Lanes in a Cell Culture Microfluidic Device With a Three-Lane Design

FIG. 9 illustrates perfusion flow in a variant cell culture microfluidic device that provides two sets of ports, one for each perfusion lane, that allows for independent pneumatic control over each lane. A check microvalve is also provided at the inlet of the second (left) receptacle in each perfusion lane. In the depicted setup, the outlet of the second receptacle is inoperative. A: Cell culture media is deposited into the two receptacles on the right, one in each perfusion lane. A cover is applied to seal the top side of the device, including the receptacles and the central lane ports. B-G: Negative pressure is applied to the bottom left pneumatic line, while the bottom right pneumatic line serves as a vent. This results in the media in the bottom perfusion lane being drawn from the bottom right receptacle past the interface with the central lane and through the check microvalve into the bottom left receptacle. H-J: Negative pressure is applied to the top left pneumatic line, while the top right pneumatic line serves as a vent. This results in the media in the top perfusion lane being drawn from the top right receptacle past the interface with the central lane and through the check microvalve into the top left receptacle.

All publications and patent applications cited in this specification are herein incorporated by reference as if each individual publication or patent application were specifically and individually indicated to be incorporated by reference. In the event of conflicting information with statements between any reference to or incorporated herein, and the present disclosure, the present disclosure will act as the guiding authority. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain variations and modifications may be made thereto without departing from the scope of the appended claims. Such variations and modifications are intended to be captured by the disclosed subject matter and/or following claims. All examples, embodiments, aspects, methods and products disclosed herein are illustrative and are not intended to be limiting in any way.

It is to be understood that any numerical value inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs. The phrase “and/or” as used herein in the specification and in the claims should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to encompass the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. As used herein, whether in the specification or the appended claims, the transitional terms “comprising”, “including”, “carrying”, “having”, “containing”, “involving”, and the like are to be understood as being inclusive or open-ended (i.e., to mean including but not limited to), and they do not exclude unrecited elements, materials or method steps. Only the transitional phrases “consisting of” and “consisting essentially of”, respectively, are closed or semi-closed transitional phrases with respect to claims and exemplary embodiment paragraphs herein. The transitional phrase “consisting of” excludes any element, step, or ingredient which is not specifically recited. The transitional phrase “consisting essentially of” limits the scope to the specified elements, materials or steps and to those that do not materially affect the basic characteristic(s) of the subject matter disclosed and/or claimed herein.

What is claimed is:

1. A perfuser comprising a relief-patterned substrate having at least two meeting surfaces for sealing with one or more covers, the relief patterning defining:
at least two pairs of medium exchange receptacles, each receptacle having: a floor; a medium outlet at or near the floor; a closed array of least one sidewall separating the floor from a first of the at least two meeting surfaces; and a gravitationally checked microvalve (GCM) for feeding medium into the receptacle at an opening of the GCM; and
a plurality of perfusion channels each having a hydraulic diameter (d_{HC}) of 0.1-1 mm for medium transport, the perfusion channels meeting the paired medium exchange receptacles at their respective medium outlets, and meeting an interface with a respective lane of a 3-raceway cell culture layer that is part of, or designed for sealed meeting with, the perfuser, whereby the medium in a first of the paired receptacles can flow through the perfusion channel to the respective lane, through the lane, and to a second receptacle paired with the first receptacle,
wherein a separation between the opening and the floor is at least 5 mm.

2. The perfuser of claim 1, wherein the GCM comprises an accretion surface on one of the at least one sidewall, or an interior pillar of the receptacle, the accretion surface surrounding the opening on at least 3 sides thereof, and adapted to guide formation and growth of a bolus of the medium and limit horizontal spread of the medium beyond the accretion surface, to draw a center of gravity of the bolus away from the first sidewall.

3. The perfuser of claim 1 further comprising a gas inlet coupling the floor of one of the paired receptacles with a valved vent, for entraining gas bubbles into the medium when the valve is open to the vent and vacuum is drawn from a headspace above the liquid in the one of the paired receptacles.

4. The perfuser of claim 1, where the substrate is composed of a thermoplastic polymer, and has relief patterning consistent with formation by injection molding.

5. The perfuser of claim 1, where the substrate further comprises at least part of a pneumatic supply lines that couples suction ports of the perfuser to the GCMs, to drive the medium transfer between the paired receptacles.

6. The perfuser of claim 5 wherein the substrate, in mounted connection with the 3-raceway cell culture layer, produces a perfusion network for cycling the medium between the paired receptacles, by which: the medium reciprocates through its lane and back during one period of the cycle; or the medium cycles through its lane flowing only from a first of the paired receptacles to a second of the paired receptacles, with the medium following a different path from the second receptacle to the first receptacle.

7. The perfuser of claim 1, wherein a bottom surface of the substrate is relief patterned to define at least part of a perfusion network that is fluidically connected to at least one of the outlets.

8. A kit comprising the perfuser of claim 1 in combination with a 3-raceway cell culture substrate, having a set of relief patterned surfaces and ports for coupling with the perfuser, to complete perfusion channels between paired receptacles.

9. The kit of claim 8, further comprising: one or more pneumatic supplies for coupling to ports of the substrate; or a controller for controlling one or more pneumatic supplies adapted to apply a relative pressure of no more than ±2 psi, such as about −0.05 to about −1 psi, preferably about −0.05 to about −0.5 psi, to induce the transfer in the perfuser.

10. A device for perfusing a cell culture
comprising: a perfuser as defined in claim 1;
a base layer having a top surface adapted to seal at least part of the bottom surface of the perfuser, wherein the base layer and the bottom surface of the perfuser together define a perfusion network that is fluidically connected to at least one of the outlets;
a cover having a bottom surface adapted to seal at least part of the top surface of the perfuser; and
a plurality of ports configured to couple to a pressure source, wherein each of the plurality of ports are fluidically connected to the plurality of receptacles, and at least one port is disposed at each opposite end of the device,
wherein the perfusion network is configured to:
provide first and second perfusion paths, each extending between two of the plurality of ports; fluidically interconnect the first pair of receptacles through the first perfusion path;
fluidically interconnect the second pair of receptacles through the second perfusion path; comprise a central region having at least one central port of the device; and
provide fluid communication between the central region and the first and second perfusion paths at a first and second interfaces, where the first and second interfaces are on opposite flanking sides of the central region.

11. The device of claim 10 wherein the perfusion network is configured such that the first and/or the second perfusion path is branched, or forms a loop.

12. The device of claims 10 wherein the bottom surface of the cover is patterned, and the top surface of the perfuser and the bottom surface of the cover together define an auxiliary network that is fluidically connected to the perfusion network.

13. The device of claim 10 wherein the perfusion network and/or auxiliary network comprises a metering receptacle and/or a micromixing receptacle.

14. The device of claim 10 wherein the perfuser and the base layer are discrete components; or the perfuser and the base layer are integrated as a single component.

15. A kit comprising a device as defined of claim 10 and a pressure source rated for supplying a maximum relative pressure of about −0.05 to about −1 psi, more preferably about −0.05 to about −0.5 psi.

16. A method of perfusing a cell culture, comprising:
a) fluidically connecting two pairs of three-phase receptacles with 2-phase, non-capillary channels and with pneumatic supply lines of a microfluidic chip;
b) providing a cell culture with at least 2 perfusion lanes, and coupling each perfusion lane with a respective pair of the receptacles; and
c) perfusing the cell culture,
wherein the pressure source is operated at a relative pressure of no more than ±2 psi to perfuse a liquid medium along the first and second perfusion paths during step c.

17. The method of claim 16 wherein the first and second perfusion paths are independently controlled.

18. The method of claim 17 wherein the first and second perfusion paths are reversed in direction at least once during step c.

19. The method of claim 17 wherein the first and second perfusion paths are provided in the same direction.

20. The method of claim 16, wherein the pressure source is operated at a relative pressure of −0.05 to −0.5 psi.
